(19) United States
(12) Patent Application Publication
(10) Pub. No.: US 2007/0199821 A1
(43) Pub. Date: Aug. 30, 2007

(54) AUTOMATED TWO-DIMENSIONAL GEL ELECTROPHORESIS

(76) Inventor: ANDREA W. CHOW, Los Altos, CA (US)

Correspondence Address: CALIPER LIFE SCIENCES, INC., 605 FAIRCHILD DRIVE, MOUNTAIN VIEW, CA, 94043-2234, US

(21) Appl. No.: 11/538,721

(22) Filed: Oct. 4, 2006

Related U.S. Application Data
(60) Provisional application No. 60/724,022, filed on Oct. 5, 2005.

(51) Int. Cl. C07K 1/26; G01N 27/00

(52) U.S. Cl. 204/451; 204/601

(57) ABSTRACT

An automated, two-dimensional gel electrophoresis technique includes methods and apparatuses for performing a functionally equivalent, automated two-dimensional gel electrophoresis process in an integrated, robotic apparatus.

The earlier effective filing data of U.S. Provisional Application Ser. No. 60/724,022, entitled “Automated Two-Dimensional Gel Electrophoresis”, filed Oct. 5, 2005 in the name of the inventor Andrea W. Chow (Attorney Docket No. 100/21600), is hereby claimed. The provisional application is also hereby incorporated by reference for all purposes as if set forth verbatim herein.

### BACKGROUND OF THE INVENTION

1. Field of the Invention

The present invention provides devices and methods for analysis of proteins. In particular, embodiments of the invention relate to the analysis of proteins using two-dimensional gel electrophoresis.

2. Description of the Related Art

Proteomics is the large-scale study of proteins, particularly their structures and functions. A variety of different analysis techniques are employed in proteomics. For example, two-dimensional (“2-D”) gel electrophoresis are used to identify the relative mass of a protein and its isoelectric point; mass spectrometry combined with reverse phase chromatography or 2-D electrophoresis is used to identify and quantify all the levels of proteins found in cells; and affinity chromatography, yeast two hybrid techniques, fluorescence resonance energy transfer (“FRET”), and Surface Plasmon Resonance (“SPR”) are used to identify protein-protein and protein-DNA binding reactions.

The first step in a typical proteomics analysis involves the preparation of a complex protein sample. Often the sample will be obtained by solubilizing proteins from sources such as tissue, cells, blood plasma, etc. It may also be desirable to remove abundant proteins such as albumin and Immunoglobulin G (“IgG”) from the sample. The sample may also benefit from desalting.

Once sample preparation is complete, 2-D gel electrophoresis is carried out. The 2-D gel electrophoresis consists of two steps. The first is isoelectric focusing (“IEF”), which separates proteins based on their relative content of acidic and basic residues. This is followed by the second, Sodium Dodecyl Sulfate Polyacrylamide Gel Electrophoresis (“SDS-PAGE”), which separates proteins according to their size (length of polypeptide chain).

Following electrophoresis, the gel may be stained (most commonly with Coomassie Brilliant Blue or silver stain), allowing visualization of the separated proteins. After staining, different proteins will appear as distinct spots within the gel. An image of the gel is then scanned, and the gel image is analyzed qualitatively. To determine the identity of a protein spot in the gel, the gel spot can be excised, subjected to proteolytic digestion, and purified. The peptide mixture that results from digestion can then be characterized using a combination of liquid chromatography (“LC”) and mass spectrometry (“MS”). The characterization process consists of fractionating the peptide mixture using one or two steps of liquid chromatography. The eluent from the chromatography steps can be either directly introduced to a mass spectrometer through electrospray ionization, or laid down on a series of small spots for later mass analysis using Matrix-Assisted Laser Desorption/Ionization (“MALDI”).

A typical proteomics analysis is time-consuming, expensive, and labor intensive. An example of a typical protocol for the 2-D gel electrophoresis analysis of a protein, provided by the Department of Biochemistry at the Medical College of Wisconsin, is posted at www.biochem.mcw.edu/protein_facility/2D.html. The first steps in that protocol, which relate to sample preparation, involves the steps of suspending protein samples in SDS and DTT (dithiothreitol; Cleland's reagent), heating the suspension for 10 min, adding rehydration buffer, incubating for 30 minutes, and centrifuging the suspension to remove insoluble debris.

The next steps in the protocol relate to performing the first dimension of separation in 2-D electrophoresis, IEF. The soluble fraction in the suspension is placed in a focusing tray, and an IPG dry strip is added to the tray. Strip rehydration is allowed to take place for 12 hours. Electrofocusing is then carried out for 8000 volt-hours. Depending on strip length and the presence of salts and detergent in the original sample, focusing could take two hours to many hours.

The next steps in the protocol relate to performing the second dimension of separation, SDS-PAGE. To prepare the IEF separated sample for second dimension separation, the IPG strip is equilibrated for 20 min in equilibrating buffer followed by 20 min in buffer containing iodoacetamide. The second dimension separation is then performed by placing the IPG strip on top of a SDS-PAGE gel box containing separation and stacking gels, and applying voltage to the gel for 1-8 hours. The protein spots on the gel are then stained using Coomassie or silver staining. The staining and subsequent destaining steps take a few hours. Finally, an image of the gel is scanned, producing a picture of the gel. The cost of producing the picture ranges from $150 to $300 per sample.

The equipment and reagents required to perform standard proteomics analyses are commercially available from a number of vendors. See, e.g. the products described on the Amersham Biosciences Proteomics Homepage at www5.amershambiosciences.com/aptrix/upp00919.nsf/content/proteomics_homepage. More specifically, there are commercially available instruments that perform individual operations in a proteomics analysis: IEF, SDS-PAGE, image scanning, spot picking, spot digestion, MALDI spot laying. Since each instrument performs only one operation, samples must be transferred manually between the individual instruments.

Recently, however, an automated workstation, the Ettan Spot Handling Workstation, has been developed that robotically transfers samples between instruments that perform spot picking, spot digestion, and MALDI spot laying. A description of the Ettan Spot Handling workstation is available on the web at http://www1.amershambiosciences.com/aptrix/upp00919.nsf/Content/Proteomics+In+Expression+Analysis+Area%5CProteomics+Robotic+Solutions%5CProteomics_Spot_Handling_Workstation. Since the Ettan Spot Handling workstation is a macro-scale system that employs standard size instruments robotic components, it is a large system that occupies a significant amount of laboratory floor space.

The present invention is directed to resolving, or at least reducing, one or all of the problems mentioned above.

### SUMMARY OF THE INVENTION

The invention, in its various aspects and embodiments, is an automated, two-dimensional gel electrophoresis technique that includes methods and apparatuses for performing a functionally equivalent, automated two-dimensional gel electrophoresis process in an integrated, robotic apparatus.

For example, in one aspect, the invention includes an integrated apparatus, that comprises, in a one embodiment, three fixtures and a robotic mechanism. The first fixture is capable of receiving a macrofluidic sample cartridge. The second fixture is capable of receiving a microfluidic isoelectric focusing cartridge and processing at least a portion of a sample deposited in the microfluidic isoelectric focusing cartridge from the macrofluidic sample cartridge to separate the sample into a plurality of first protein fractions having different isoelectric points. The third fixture is capable of receiving a microfluidic separation cartridge and processing a first protein fraction deposited in the microfluidic separation cartridge from the isoelectric focusing cartridge and processing the first protein fraction to separate the first protein fraction into a plurality of second protein fractions having different sizes. The robotic mechanism capable of robotically transferring the sample from the microfluidic sample cartridge to the microfluidic isoelectric focusing cartridge and the first protein fraction from the microfluidic isoelectric focusing cartridge to the microfluidic separation cartridge.

In another embodiment, the integrated apparatus comprises two means. The first means is for receiving a protein-containing sample, microfluidically isoelectrically focusing the proteins of the sample into a plurality of first protein fractions, and microfluidically separating one of the plurality of first protein fractions into a plurality of second protein fractions by size. The second means for robotically handling the fluids used by the receiving, isoelectrically focusing, and separating means.

In another aspect, the invention includes a method, comprising: providing a sample comprising a plurality of proteins; robotically transferring the sample to a first microfluidic device; isoelectrically focusing the proteins of the provided sample to separate the proteins into a plurality of first protein fractions having different isoelectric points; robotically transferring a first protein fraction to a second microfluidic device; and separating the first protein fraction into a plurality of second protein fractions.

### BRIEF DESCRIPTION OF THE DRAWINGS

The invention may be understood by reference to the following description taken in conjunction with the accompanying drawings, in which like reference numerals identify like elements, and in which:

FIG. 1 depicts an integrated macro and microfluidic platform (“IMMP”) in accordance with one aspect of the present invention in the context of its use in accordance with another aspect of the present invention;

FIG. 2 schematically depicts a free flow isoelectric focusing microfluidic device for use in the embodiment of FIG. 1;

FIG. 3 schematically depicts one embodiment of a protein separation microfluidic device for use in the embodiment of FIG. 1;

FIG. 4 schematically depicts a second embodiment of a protein separation microfluidic device alternative to that in FIG. 3 for use in the embodiment of FIG. 1; and

FIG. 5 illustrates one embodiment of a method practiced in accordance with the present invention.

While the invention is susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

In its various aspects and embodiments, the present invention provide methods and apparatuses for performing 2-D gel electrophoresis in a platform that integrates macrofluidic and microfluidic technologies. Embodiments of the invention provide faster and less labor-intensive workflows than conventional 2-D methods and apparatuses.

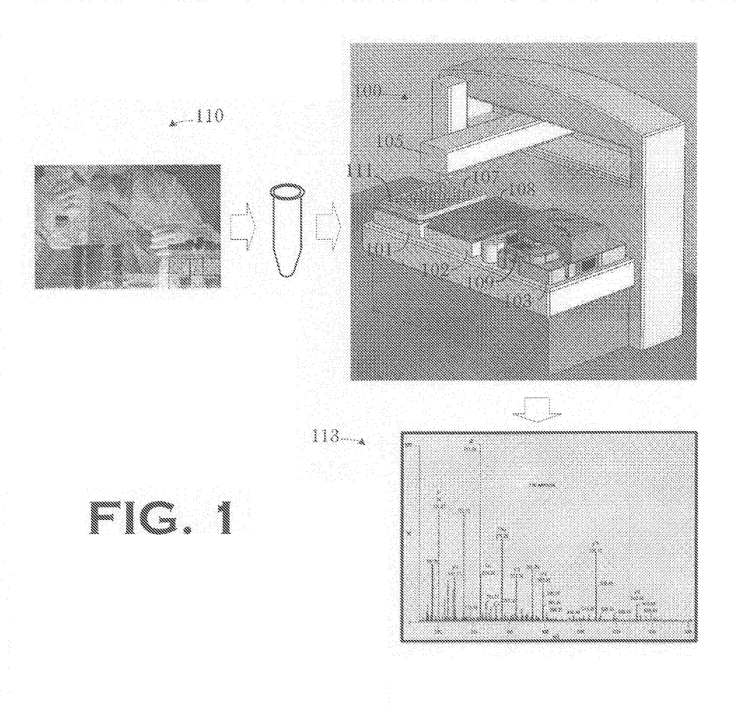

An example of an integrated macro and microfluidic platform (“IMMP”) 100 in accordance with the invention is shown in FIG. 1. The platform 100 comprises three fixtures 101-103 adapted to receive three different types of removable cartridges 107-109. A robotic mechanism, e.g., an arm, 105 transfers reagents and sample solutions between the three cartridges 107-109 on platform 100. The three fixtures 101-103 comprise at least a portion of abase 112.

The first fixture 101 is adapted to receive a cartridge 107 containing a plurality of wells 111 (only one indicated) configured to receive the various reagents (not shown) required to carry out 2-D gel electrophoresis, along with a purified protein sample solution (also not shown). The cartridge 107 could be a standard microtiter plate, or a standard fixture configured to received vials or flasks. The protein sample solution placed into the cartridge 107 received by the first fixture 101 will typically be produced by solubilizing proteins from sources such as tissue, cells, blood plasma, etc., removing undesired abundant proteins such as albumin and IgG from the resulting solution, and in some cases desalting the solution.

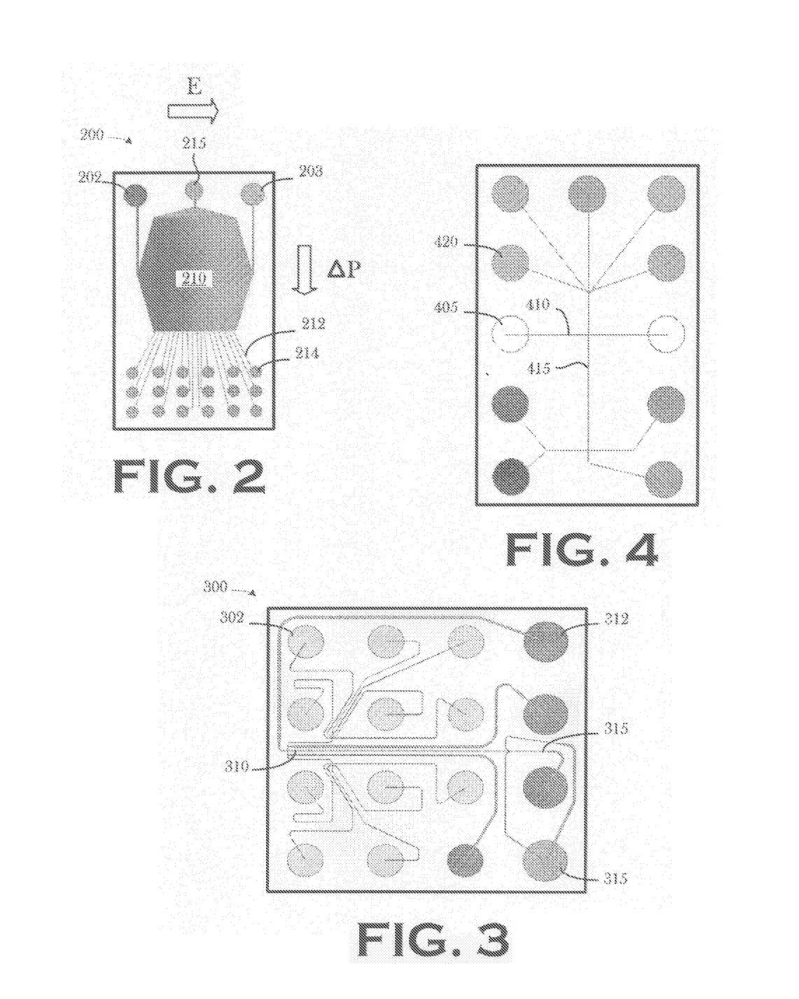

The cartridge 108 received by the fixture 102 is a microfluidic device configured to carry out the first dimension of 2-D gel electrophoresis analysis, IEF. In addition, after the IEF process is complete, the cartridge 108 received in the fixture 102 may also further prepare the processed sample for transfer to an SDS-PAGE process. One embodiment for the cartridge 108 is shown in FIG. 2.

A free flow IEF microfluidic device 200 for use in accordance with once aspect of the invention is shown in FIG. 2. The device 200 in FIG. 2 is based on the technology discussed in:
U.S. Pat. No. 5,599,432, entitled “Device and a Method for the Electrophoretic Separation of Fluid Substance Mixtures”, issued Feb. 4, 1997, to Ciba-Geigy Corporation as assignee of the inventors Andreas Manz and Carlos S. Effenhauser; and
U.S. Pat. No. 5,180,480, entitled “Apparatus for the Preparation of Samples, Especially for Analytical Purposes”, issued Jan. 19, 1993, to Ciba-Geigy Corporation as assignee of the inventor Andreas Manz.
   Both of these patents are incorporated by reference in their entirety for all purposes as if set forth verbatim herein.

In the device 200 shown in FIG. 2, ampholytes in the reservoirs 202, 203 are chosen so that a desired pH range spans horizontally across the large chamber 210 in the device 200 when electric field E is applied. When the desired pH range is present, the protein fractions (not shown) introduced into the large chamber 210 from sample reservoir 215 will segregate across the width of the chamber 210 according to their isoelectric points (“pI”). A plurality of channels 212 (only one indicated) across the bottom edge of the chamber 210 collect protein fractions at different locations across the width of the chamber as a pressure gradient ΔP is applied, and direct the individual fractions collected at those various locations to different wells 214 (only one indicated) on the microfluidic device 200. As previously discussed, the fractions in those wells 214 can be prepared to undergo an SDS-PAGE process through the addition of reagents (not shown), and the application of heat for an appropriate incubation time. Also, as previously discussed, desired fractions in the wells 214 can be transferred to a fraction collection microfluidic device where they are prepared to undergo LC/MS analysis.

Returning to FIG. 1, the cartridge 109 received in the fixture 103 is a microfluidic device configured to perform the second dimension of 2-D gel electrophoresis, separation by size, by carrying out a process that is functionally equivalent to a standard SDS-PAGE process. The separation process carried out in the cartridge 109 received in the fixture 103 could be an SDS-PAGE process, or any other process that separates proteins by size, including separation processes that do not employ a gel. Furthermore, the cartridge 109 received in the fixture 103 could perform other functions beyond size separation, such as fraction collection and protein digestion. Two embodiments for the cartridge 109 are shown in FIG. 3 and in FIG. 4.

Referring now to FIG. 3, a microfluidic device 300 configured to carry out SDS-PAGE separation of the components in a protein fraction suitable for use in the present invention is shown. The operation of the device 300 is described in U.S. Pat. No. 6,475,364, entitled “Methods, Devices and Systems for Characterizing Proteins”, issued Nov. 5, 2002, to Caliper Technologies Corp. as assignee of the inventors Robert S. Dubrow, et al. This patent is incorporated by reference in its entirety for all purposes as if set forth verbatim herein.

As described in the '364 patent, the device 300 in FIG. 3 is configured to receive a plurality of samples in a plurality of wells 302 (only one indicated), and to separate the components of each of those samples by subjecting them to an SDS-PAGE separation process by passing the samples through a separation channel 310. The reservoirs 312 (only one indicated) and 313 are used for gel priming and dilution buffer, respectively. The separation channel 310 ends in a detection region 315, where the size-separated components of the sample are quantitatively detected. The quantitative peak data collected in detection region 315 can be converted into a format that provides the same information as the image data collected from a conventional gel. One technique for doing so is disclosed in U.S. Pat. No. 6,430,512, entitled, “Software for the Display of Chromatographic Separation Data,” issued Aug. 6, 2002, to Caliper Technologies Corp. as assignee of the inventor Steven J. Gallagher. This patent is incorporated by reference in its entirety for all purposes as if set forth verbatim herein.

Turning now to FIG. 4, a second fraction collection microfluidic device 400 suitable for use in the present invention is shown. The basic principles behind the operation of the fraction collection device 400 are described in U.S. Pat. No. 5,858,195, entitled “Apparatus and Method for Performing Microfluidic Manipulations for Chemical Analysis and Synthesis”, issued Jan. 12, 1999, to Lockheed Martin Energy Research Corporation as assignee of the inventor J. Michael Ramsey. This patent is incorporated by reference in its entirety for all purposes as if set forth verbatim herein.

As previously discussed, a sample comprising an IEF separated protein fractions from the IEF microfluidic device is placed into a sample well 405 (only one indicated) in the fraction collection microfluidic device 400. Using the principles of electrokinetic flow control described in the '195 patent, a stream of sample is directed to flow through a channel 410 extending from the sample reservoir 405 across a separation channel 415. A portion of the sample stream is directed into the separation channel 415, where it undergoes a capillary electrophoresis separation process, separating the sample into a plurality of components (not shown). The capillary electrophoresis process may or may not involve the use of a gel.

Components of interest can be selectively directed into individual fraction collection wells 420 (only one indicated) at the end of the separation channel 415. Thus, the fraction collection microfluidic device 400 can take an isoelectric protein fraction from a specific sample well 405 on the IEF microfluidic device, separate the isoelectric protein fraction into multiple molecular weight fractions with no or very little gel in the fractions. Trypsin digestion of the multiple molecular weight fractions can be performed in the fraction collection wells 420 by adding bead-bound trypsin to the wells 420 after fractionation is complete.

Returning to FIG. 1, the preparation process for the sample is schematically represented by the image 110, and the transfer of the prepared protein solution into the cartridge received by fixture 101 is schematically represented by image 120. Note that in other embodiments of the invention, sample preparation could take place in another cartridge in a platform similar to platform 100. The cartridge received by fixture 101 could be configured as a kit, where the cartridge received by fixture 101 is pre-packaged to contain all of the required reagents. Alternatively, the required reagents could be manually placed into the appropriate wells in the cartridge.

The sample is then subjected to a protein separation process functionally equivalent to a 2D gel electrophoresis process. The functionally equivalent process would comprise the steps of loading a sample from the cartridge 107 received in the fixture 101 into the cartridge 108 received in the fixture 102, where the cartridge 108 received in fixture 102 is a microfluidic free flow IEF device. The microfluidic free flow IEF device is configured so that protein fractions having different isoelectric points can be collected in a series of wells on the microfluidic device. Next, SDS buffer, with or without denaturant, is added to those wells, and the microfluidic device (i.e., the cartridge 108) in fixture 102 is heated, so that the reagents added to the wells can interact with protein fractions sufficiently so that the protein fractions are prepared to be subjected to a gel separation process. Note that in the embodiment of FIG. 1, reagents such as SDS buffer and denaturant can be transferred into the wells in the device received in fixture 102 by the robotic arm 105 from wells in the cartridge received in fixture 101, using standard liquid handling technology. The microfluidic device received in fixture 102 could be heated by a heating element (not shown) within instrument 100 that is in thermal contact with fixture 102.

After the protein fractions in the microfluidic device received in fixture 102 have been suitably prepared to be subjected to a gel separation process, protein fractions can be transferred to a microfluidic device (e.g., one of the devices 300, 400 in FIG. 3, FIG. 4) received in fixture 103 that is configured to carry out SDS-PAGE separation of the components in each fraction. Transfer of the fractions from the wells in the microfluidic device received in fixture 102 to the microfluidic device received in fixture 103 could be effectuated by robotic arm 105. As a result of the SDS-PAGE being carried out on a microfluidic device, the results of the size separation of the protein fractions will inherently be quantitative in nature. Thus, the time required to analyze the quantitative results, e.g., to identify protein fractions of interest, will be around fifteen minutes.

Those protein fractions can be selectively collected from the wells of the IEF microfluidic device received in fixture 102, and transferred to a fraction collection microfluidic device received within fixture 103. The collection process will typically take around one or two hours. Note that the modular features of platform 100 allow the SDS-PAGE microfluidic device previously received in fixture 103 to be removed and replaced with a different cartridge configured to perform fraction collection on a microfluidic device.

Once the appropriate IEF-separated protein fractions are transferred to the fraction collection microfluidic device by robotic arm 105, the fraction collection microfluidic device can separate the components of a protein fraction by size, and divert desirable components into separate wells on the fraction collection microfluidic device. Those components are equivalent to the protein spots generated in a conventional 2-D gel electrophoresis process. Within the wells of the fraction collection microfluidic device, the individual components can be digested by adding trypsin-bound beads into the wells, and incubating the components with the trypsin-bound bead for an appropriate incubation period. The total digestion process takes around fifteen minutes. After the incubation period, the individual components are ready to be subjected to standard LC/MS analysis. The results can then be reported in conventional fashion, as represented by the graphic 113 in FIG. 1.

The workflow required to perform a proteomics analysis on an apparatus in accordance with the invention is therefore less labor intensive and time consuming than the workflow required to perform the same analysis on conventional equipment. As previously discussed, the sample preparation process schematically represented in image 110 in FIG. 1 is essentially then same as the sample preparation process in a conventional workflow. In an apparatus in accordance with the invention, however, a process that is functionally equivalent to a conventional 2-D gel electrophoresis process can be performed much rapidly than a conventional 2-D gel electrophoresis process. While a conventional 2-D gel electrophoresis process can take anywhere from around 4-13 hours total, even after the sample has been rehydrated, a functionally equivalent process can be performed in an apparatus in accordance with the invention in around 2-3 hours.

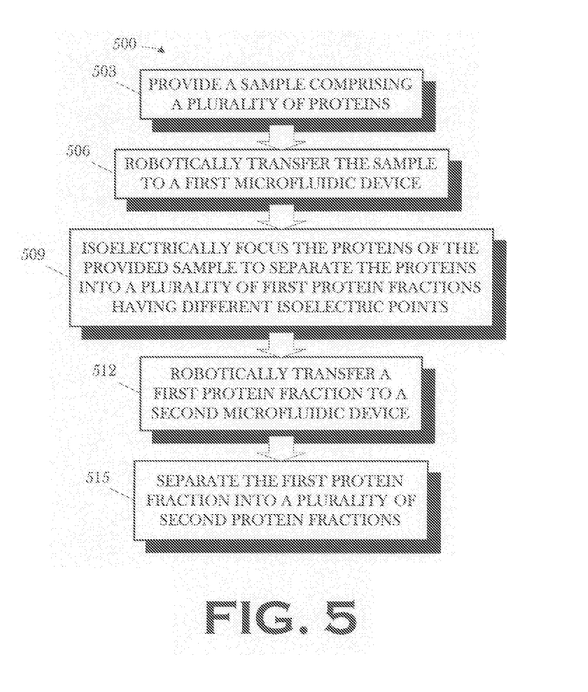

Thus, in one aspect, the invention comprises a method 500, illustrated in FIG. 5, including:
providing (at 503) a sample comprising a plurality of proteins;
robotically transferring (at 506) the sample to a first microfluidic device;
isoelectrically focusing (at 509) the proteins of the provided sample to separate the proteins into a plurality of first protein fractions having different isoelectric points;
robotically transferring (at 512) a first protein fraction to a second microfluidic device; and
separating (at 515) the first protein fraction into a plurality of second protein fractions.
   In a second aspect, the invention comprises an apparatus, e.g., the apparatus 100 in FIG. 1, comprising:
a first fixture (e.g., the first fixture 101) capable of receiving a macrofluidic sample cartridge (e.g., the cartridge 107);
a second fixture (e.g., the first fixture 102) capable of receiving a microfluidic isoelectric focusing cartridge (e.g., the cartridge 108) and processing at least a portion of a sample deposited in the microfluidic isoelectric focusing cartridge from the macrofluidic sample cartridge to separate the sample into a plurality of first protein fractions having different isoelectric points;
a third fixture (e.g., the first fixture 103) capable of receiving a microfluidic separation cartridge (e.g., the cartridge 109) and processing a first protein fraction deposited in the microfluidic separation cartridge from the isoelectric focusing cartridge and processing the first protein fraction to separate the first protein fraction into a plurality of second protein fractions having different sizes; and
a robotic mechanism (e.g., the arm 105) capable of robotically transferring the sample from the microfluidic sample cartridge to the microfluidic isoelectric focusing cartridge and the first protein fraction from the microfluidic isoelectric focusing cartridge to the microfluidic separation cartridge.
   Note, however, that the invention admits variation in the implementation of the apparatus and the method. For example, the first, second and third fixtures are, by way of example and illustration, just some of the means for receiving a protein-containing sample, microfluidically isoelectrically focusing the proteins of the sample into a plurality of first protein fractions, and microfluidically separating one of the plurality of first protein fractions into a plurality of second protein fractions by size. Similarly, the robotic mechanism is, by way of example and illustration, but one means for robotically handling the fluids used by the receiving, isoelectrcially focusing, and separating means.

Apparatuses and methods in accordance with the invention provide a higher degree of automation than conventional 2-D gel electrophoresis apparatuses and methods. Although in embodiments of the invention the process of preparing a complex protein sample is performed manually, as in conventional 2-D gel electrophoresis workflows, the steps of performing the 2-D gel electrophoresis process (IEF followed by SDS-PAGE) and creating a gel image are completely automated. Although the process of analyzing the 2-D gel image and deciding which protein fractions merit further analysis require manual intervention in embodiments of the invention, the processes of collecting the desired protein fractions and digesting those fractions can be automated are automated. The steps in the proteomics analysis downstream of 2-D gel electrophoresis, starting with the step of transferring desired fractions to instruments that perform LC/MS analysis, are performed in the conventional manner even when apparatuses and methods in accordance with the invention are used to carry out the 2-D gel electrophoresis portion of the proteomics analysis.

The benefits of employing embodiments of the invention to carry out the 2-D gel electrophoresis process are a faster and less labor intensive workflow compared to conventional 2-D gel electrophoresis workflow, lower cost of equipment compared to existing fully automated spot handling workstations for lower throughput laboratories, higher sensitivity to low abundance proteins than 2-D gel electrophoresis processes that are carried out on a single microfluidic device, and a minimization of the potential loss of proteins through the minimization of the number of sample transfer steps. As previously described, embodiments of the invention can involve only three transfers of protein samples: from the sample vial to the IEF microfluidic device, from the IEF microfluidic device to the fraction collection/digestion device, and from the fraction collection/digestion device to the LC/MS instrument.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

This concludes the detailed description. The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. An integrated apparatus, comprising:
a first fixture capable of receiving a macrofluidic sample cartridge;
a second fixture capable of receiving a microfluidic isoelectric focusing cartridge and processing at least a portion of a sample deposited in the microfluidic isoelectric focusing cartridge from the macrofluidic sample cartridge to separate the sample into a plurality of first protein fractions having different isoelectric points;
a third fixture capable of receiving a microfluidic separation cartridge and processing a first protein fraction deposited in the microfluidic separation cartridge from the isoelectric focusing cartridge and processing the first protein fraction to separate the first protein fraction into a plurality of second protein fractions having different sizes; and
a robotic mechanism capable of robotically transferring the sample from the microfluidic sample cartridge to the microfluidic isoelectric focusing cartridge and the first protein fraction from the microfluidic isoelectric focusing cartridge to the microfluidic separation cartridge.

2. The integrated apparatus of claim 1, further comprising at least one of the macrofluidic sample cartridge, the microfluidic isoelectric focusing cartridge, and the microfluidic separation cartridge.

3. The integrated apparatus of claim 2, wherein the macrofluidic sample cartridge comprises one of a microtiter plate, fixture configured to receive a plurality of vials, or a fixture configured to receive a plurality of flasks.

4. The integrated apparatus of claim 2, wherein the microfluidic isoelectric focusing cartridge comprises a microfluidic free flow isoelectric focusing device.

5. The integrated apparatus of claim 2, wherein the microfluidic isoelectric focusing cartridge comprises a device body defining:
a sample well into which a sample may be deposited;
a plurality of ampholyte wells into which a plurality of ampholytes may be deposited;
a chamber into which the sample and the ampholytes may feed responsive to a pressure gradient for segregation by their respective isoelectric points responsive to the imposition of an electric field across the chamber; and
a plurality of fraction wells into which the segregated first protein factions may be collected.

6. The integrated apparatus of claim 2, wherein the microfluidic separation cartridge comprises a device body defining:
a plurality of sample wells into which a plurality of samples may be deposited;
a dilution buffer well into which a dilution buffer may be deposited;
a separation channel into which the sample, and dilution buffer may be introduced and to separate the components of the sample and in which the separated component samples may be quantitatively detected.

7. The integrated apparatus of claim 2, wherein the microfluidic separation cartridge comprises a device body defining:
a sample well into which a sample may be deposited;
a separation channel into which the sample may be directed by an electrokinetic flow control for a capillary electrophoresis separation; and
a plurality of collection wells into which the separated sample components may be collected.

8. The integrated apparatus of claim 1, wherein the second fixture is further capable of processing a first protein fraction to prepare it for the protein separation.

9. The integrated apparatus of claim 1, wherein the second fixture is further capable of heating the first protein fraction.

10. The integrated apparatus of claim 1, wherein the third fixture is capable of performing a Sodium Dodecyl Sulfate Polyacrylamide Gel Electrophoresis process to separate the proteins.

11. The integrated apparatus of claim 1, wherein the third fixture is further capable of processing the second protein fraction for fraction collection or protein digestion.

12. The integrated apparatus of claim 1, wherein the first, second and third fixtures comprise at least a portion of a base.

13. An integrated apparatus, comprising:
means for receiving a protein-containing sample, microfluidically isoelectrically focusing the proteins of the sample into a plurality of first protein fractions, and microfluidically separating one of the plurality of first protein fractions into a plurality of second protein fractions by size; and
means for robotically handling the fluids used by the receiving, isoelectrcially focusing, and separating means.

14. The integrated apparatus of claim 13, wherein the receiving, isoelectrcially focusing, and separating means includes:
a first fixture capable of receiving a macrofluidic sample cartridge;
a second fixture capable of receiving a microfluidic isoelectric focusing cartridge in which the proteins of the sample are microfluidically isoelectrically focused into a plurality of first protein fractions;
a third fixture capable of receiving a microfluidic separation cartridge in which a first protein fraction is microfluidically separated into a plurality of second protein fractions; and
a robotic mechanism capable of robotically transferring the sample from the microfluidic sample cartridge to the microfluidic isoelectric focusing cartridge and the first protein fraction from the microfluidic isoelectric focusing cartridge to the microfluidic separation cartridge.

15. The integrated apparatus of claim 14, further comprising at least one of the macrofluidic sample cartridge, the microfluidic isoelectric focusing cartridge, and the microfluidic separation cartridge.

16. The integrated apparatus of claim 14, wherein the second fixture is further capable of processing a first protein fraction to prepare it for the protein separation.

17. The integrated apparatus of claim 14, wherein the second fixture is further capable of heating the first protein fraction.

18. The integrated apparatus of claim 14, wherein the third fixture is capable of performing a Sodium Dodecyl Sulfate Polyacrylamide Gel Electrophoresis process to separate the proteins.

19. The integrated apparatus of claim 14, wherein the third fixture is further capable of processing the second protein fraction for fraction collection or protein digestion.

20. The integrated apparatus of claim 13, wherein the robotic fluid handling means comprises a robotic arm.

21. A method, comprising:
providing a sample comprising a plurality of proteins;
robotically transferring the sample to a first microfluidic device;
isoelectrically focusing the proteins of the provided sample to separate the proteins into a plurality of first protein fractions having different isoelectric points;
robotically transferring a first protein fraction to a second microfluidic device; and
separating the first protein fraction into a plurality of second protein fractions.

22. The method of claim 21, wherein providing the sample includes manually providing the sample.

23. The method of claim 21, wherein robotically transferring the sample to the first microfluidic device includes:
robotically extracting the sample from a macrofluidic source; and
robotically depositing the extracted sample in a well of the first microfluidic device.

24. The method of claim E20, wherein the first microfluidic device is a microfluidic free flow isoelectric focusing device.

25. The method of claim 21, wherein isoelectrically focusing the provided sample includes:
mixing the provided sample and an ampholyte to form a mixture; and
imposing an electric field across the mixture to segregate the proteins in the sample by their respective isoelectric points.

26. The method of 21, wherein robotically transferring the first protein fraction to the second microfluidic device includes:
robotically extracting the first protein fraction from a well in the first microfluidic device; and
robotically depositing the first protein fraction in a respective well of a microfluidic device.

27. The method of claim 21, wherein separating the first protein fraction includes performing a Sodium Dodecyl Sulfate Polyacrylamide Gel Electrophoresis separation.

28. The method of claim 21, wherein separating the first protein fraction includes performing a capillary electrophoresis separation.
